(19) United States
(12) Patent Application Publication
(10) Pub. No.: US 2009/0306024 A1
(43) Pub. Date: Dec. 10, 2009

(54) Combination preparations of o-acetylsalicylic acid salts

(76) Inventors: Wolfram Ledwoch, Langenfeld (DE); Claus Gert Haase, Recklinghausen (DE); Gerhard Franckowiak, Wuppertal (DE); Heike Wagner, Heidelberg (DE); Yutaka Hayauchi, Leverkusen (DE)

Correspondence Address: Barbara A. Shimei;Director, Patents & Licensing, Bayer HealthCare LLC - Pharmaceuticals, 555 White Plains Road, Third Floor, Tarrytown, NY, 10591, US

(21) Appl. No.: 12/090,179

(22) PCT Filed: Oct. 4, 2006
(86) PCT No.: PCT/EP2006/009573

§ 371 Date: Nov. 20, 2008

(30) Foreign Application Priority Data
Oct. 15, 2005 (DE) 10200549293.2

(51) Int. Cl. A61K 31/60; A61P 9/00

(52) U.S. Cl. 514/161

(57) ABSTRACT

The present invention relates to a fixed combination comprising a salt of o-acetylsalicylic acid with a basic amino acid as component A and an HMG-CoA reductase inhibitor as component B, a medicament comprising this combination and a process for its production.

The present invention relates to a fixed combination comprising a salt of o-acetylsalicylic acid with a basic amino acid as component A and an HMG-CoA reductase inhibitor as component B, a medicament comprising this combination and a process for its production.

The analgesic action of o-acetylsalicylic acid has been used therapeutically for a long time. Thus, o-acetylsalicylic acid is employed as an analgesic, antipyretic, antirheumatic, and as a nonsteroidal anti-inflammatory agent, for example for the treatment of arthritis, neuralgia and myalgia. In addition, o-acetylsalicylic acid, however, is also employed for secondary prevention (Antiplatelet Trialists' Collaboration: Collaborative overview of randomized trials of antiplatelet therapy. I: Prevention of death, myocardial infarction, and stroke by prolonged antiplatelet therapy in various categories of patients. Brit. Med. J. 1994, 308, 81-106) and for the primary prevention of cardiovascular diseases (U.S. Preventive Services Task Force: Aspirin for the primary prevention of cardiovascular events: recommendation and rationale. Ann. Intern. Med. 2002, 136, 157-160).

Known salts of o-acetylsalicylic acid (ASA) are, inter alia, salts of o-acetylsalicylic acid with basic amino acids. By means of the oral administration, for example, of the lysine salt of o-acetylsalicylic acid (D,L-lysine o-acetylsalicylate) an influx of the active substance can be achieved which almost corresponds to the blood level curve of a bolus injection. This is described in the literature [Ch. Raschka, H. J. Koch, Perfusion 6 (2000), 13th year, Verlag PERFUSION, Nuremberg]. The active substance dissolves extremely rapidly and is immediately absorbed by the body.

Therapeutically, for example, the salt of o-acetylsalicylic acid (ASA) with the amino acid lysine is employed. The most prevalent medicament containing D,L-lysine o-acetylsalicylate is a presentation form for parenteral administration additionally comprising glycine. It is on the market under the name Aspisol® (up to mid-2005). The glycine is added to the ASA lysinate solid, such that a mixture of D,L-lysine o-acetylsalicylate and glycine is present.

In WO 02/005782 and WO 03/059323, salts of o-acetylsalicylic acid with basic amino acids are described which have increased stability. The salts are prepared by a special process and are distinguished in a particle size distribution measured using a Malvern 2600D apparatus under standard conditions by an average particle size above a particle size of 160 μm and a proportion of more than 60% of the particles having a particle size in a range from 100 to 200 μm. They can have a certain content of added glycine which, however, has no influence on the properties of the o-acetylsalicylate, in particular on its stability. HMG-CoA reductase inhibitors, which include the class consisting of the statins, are well-known to the person skilled in the art as lipid-lowering agents. The action of the statins in the primary prevention of cardiovascular diseases by lowering the cholesterol level (Heart Protection Study of cholesterin lowering with simvastatin, LANCET 360, 2002, 7-22) and also in secondary prevention (The Scandinavian Simvastatin Survival Study, The Lancet 344, 1994, 1383-1389) is described by way of example of simvastatin. WO 99/47123 discloses the combination of o-acetylsalicylic acid (ASA) with statins, in particular pravastatin, lovastatin, simvastatin, atorvastatin, fluvastatin or cerivastatin, and the use of this combination for lowering the cholesterol level in the blood and for the prevention and treatment of cardiovascular diseases.

WO 99/47123 starts out from the chemical incompatibility between o-acetylsalicylic acid and statins and describes a special pharmaceutical formulation comprising ASA and a statin, in particular pravastatin, which reduces the interaction between o-acetylsalicylic acid and statin by both components being present separately from one another, for example in different layers.

The low stability of o-acetylsalicylic acid and its salts is to be attributed to a release of salicylic acid known to the person skilled in the art. The presence of free salicylic acid in pharmaceutical preparations, however, is undesirable and therefore to be restricted to a low, acceptable value.

The object of the present invention is the provision of a pharmaceutical presentation form which is adequately stable and simple to prepare, and which is suitable for releasing o-acetylsalicylic acid and statin.

Surprisingly, it has now been found that the presentation forms according to the invention comprising a salt of o-acetylsalicylic acid with a basic amino acid as component A and statins as component B are adequately stable and simple to prepare.

The present invention relates to a fixed combination comprising a salt of o-acetylsalicylic acid with a basic amino acid as component A and an HMG-CoA reductase inhibitor as component B.

The basic amino acid according to the invention suitable as a component of the salt of o-acetylsalicylic acid can occur in the L or in the D configuration or alternatively as a mixture of D and L form. The term “amino acid” designates according to the invention in particular the naturally occurring α-amino acids, but moreover also comprises their homologues, isomers and derivatives. Enantiomers can be mentioned as an example of isomers. Derivatives can be, for example, amino acids provided with protective groups. Typical examples of basic amino acids which may be mentioned are: lysine, arginine, ornithine, diaminobutyric acid. The salt of o-acetylsalicylic acid with lysine is particularly highly suitable. Here, the lysine can be present in the D or L form, or as a mixture of D and L form. Protective groups for amino acids are known to the person skilled in the art and are described in T. W. Greene, P. G. Wuts, Protective Groups in Organic Synthesis, 3^{rd} ed., John Wiley, New York, 1999.

With respect to details of HMG-CoA reductase inhibitors—the abbreviation “HMG-CoA” in this case stands for “3-hydroxy-3-methylglutarylcoenzyme A”—and in particular of the statins, reference is made to the discussions in Drugs of the Future 1994, 19(6), pages 537-541, and 1995, 20(6), page 611, and 1996, 21(6), page 642. A further survey of HMG-CoA reductase inhibitors is contained in Pharmazic in unscrer Zeit [Pharmacy in our Time], 28th year, No. 3, pages 147-152 (1999).

HMG-CoA reductase inhibitors as component B in the context of the invention in general represent classes of substance mentioned in the prior art under this term. Preferred classes of substances mentioned under this term are statins, as are described, for example, in EP 247 633, U.S. Pat. No. 5,006,530, EP 33 538, U.S. Pat. No. 4,346,227, EP 22 478, EP 491 226, EP 325 130 or EP 114 027.

Preferred substances from the group consisting of the statins are pravastatin, lovastatin (MEVACOR®; U.S. Pat. No. 4,231,938), simvastatin (ZOCOR®; U.S. Pat. No. 4,444,784), atorvastatin (LIPITOR®; U.S. Pat. No. 5,273,995), fluvastatin (LESCOL®; U.S. Pat. No. 5,354,772), itavastatin (“NK-104”, see EP 0304 063), pitavastatin, rosuvastatin and ZD-4522 (see EP 521 471; systematic name: (+)-(3R,5S)-bis(7-(4- (4-fluorophenyl)-6-isopropyl-2-(N-methyl-N-methanesulphonylamino)pyrmidin-5-yl)-3,5-dihydroxy-6(E)-heptenoic acid, calcium salt) or cerivastatin (U.S. Pat. No. 5,177,080).

Particularly preferred substances are atorvastatin, pravastatin, lovastatin and simvastatin. A very particularly preferred substance is simvastatin.

Statins can be present in the form of their esters or lactones or as a carboxylic acid or salts of the carboxylic acid. In the case of cerivastatin, the sodium salt (cerivastatin sodium) is particularly preferably employed. Preparation processes for the HMG-CoA reductase inhibitors are known to the person skilled in the art.

### Medicaments:

Surprisingly, it has been found that it is possible to process the two components A and B in the form of a fixed combination to give a stable formulation, in which both components A and B are present together in the same layer in a common matrix.

“Fixed combination” within the meaning of this invention is understood as meaning those pharmaceutical forms in which the components are present together in a fixed quantitative ratio. A fixed combination of this type can be produced, for example, as a tablet, film-coated tablet, sugar-coated tablet, granules, capsule or powder.

The presentation form comprising the fixed combination according to the invention of the components A and B can optionally be obtained with addition of suitable excipients by production, for example, of a mixture of both components and direct filling, for example, into sachets or capsules or formulation to give tablets.

Pharmaceutical excipients, as are familiar to the person skilled in the art, are also described, for example, in the following Handbook: “Handbook of Pharmaceutical Excipients”, Wade, A. & Weller, P. J., American Pharmaceutical Association, Washington, 2nd edition 1994.

Excipients are those which are pharmaceutically accepted and physiologically innocuous, for example fillers, disintegrants and glidants. Examples of the excipient groups mentioned can be, as fillers, cellulose derivatives (e.g. microcrystalline cellulose), native and modified starches (e.g. potato starch), sugars (e.g. lactose), sugar alcohols (e.g. mannitol, sorbitol), inorganic fillers (e.g. calcium phosphate, magnesium oxide) and buffer substances; as disintegrants, starch derivatives (e.g. crosslinked sodium carboxymethyl starch, sodium starch glycolate), cellulose derivatives (e.g. crosslinked sodium carboxymethylcellulose) and crosslinked polyvinylpyrrolidone; as glidants (understood here as a generic term for flow-regulating agents/lubricants/mould release agents): magnesium stearate, calcium stearate, stearic acid, talc and highly disperse silica.

In order to guarantee a uniform dispersion of the two substances in the mixture, the two components A and B are first mixed with one another and the excipients are intermixed thereto in stages. During production and storage, if necessary attention is to be paid to appropriate protection against moisture.

The present invention also relates to further processing of the active substance/excipient mixture already described to give granules. According to the invention, the known conventional processes are suitable for granulation. A preferred granulation process is dry granulation, in particular roll compaction. Additionally to the excipients already mentioned, the use of binders, for example sugars, sugar alcohols, starches, cellulose derivatives, alginates, pectins, polyethylene glycols and polyvinylpyrrolidone can take place. The addition of antistatics is possible. Here, polyethylene glycols, sodium lauryl sulphate, highly disperse silica/alumina or dicalcium phosphate are possibilities. The granules produced can be filled directly, for example in sachets or capsules or can be formulated to give tablets. If necessary, attention is to be paid to adequate protection against moisture during production and storage.

Moreover, the production of the fixed combination according to the invention can also be carried out by the production of two mixtures which are different from one another or granules comprising the components A and B and optionally further excipients, which are combined in an additional process step and subsequently filled, for example, into sachets or capsules or formulated to give a tablet. Each individual production step can be characterized by the addition of suitable excipients. The preferred granulation process for component A is in this case a dry granulation process, in particular roll compaction. Component B can be granulated either in dry or wet form. In the case of wet granulation, water, a suitable organic solvent such as, for example, ethanol, acetone or isopropanol, or a mixture of water with a suitable organic solvent can be employed. The addition of hydrophilizing agents in the form of surfactants, for example sodium lauryl sulphate or polysorbates, is possible. When using a wet granulation process, attention is to be paid to a low residual moisture of the granules. After the combination of the granules of the components A and B, if necessary an adequate protection against moisture is to be guaranteed in the following process steps (finishing and storage).

The present invention likewise relates to the production of a fixed combination using melt extrusion, a process which likewise follows standard procedures. Here, component A can be employed in the form of the active substance/excipient mixture already described above or granules thereof, while component B is first processed to give a melt extrudate. For the process of melt extrusion, the use of at least one polymer, plasticizer and/or carrier is necessary. Suitable polymers are, for example, hydroxypropylcellulose and polyvinylpyrrolidone. Suitable plasticizers are, for example, triethyl citrate, benzoic acid and succinic acid. The term vehicles is understood here as meaning excipients such as, for example, sugar alcohols, in particular mannitol, microcrystalline cellulose and crosslinked sodium carboxymethylcellulose. The melt extrudate obtained comprising component B is comminuted after production thereof, preferably to a particle size of less than 0.315 mm. After adding together the melt extrudate comprising component B and the active substance/excipient mixture or granulated mixture comprising the component A, direct filling as granules, for example in sachets or capsules or compaction to give tablets can take place. Addition of lubricant is to be recommended under these conditions. After combination of the active substance/excipient preparations of the components A and B in the form of the mixing step, if necessary attention is to be paid to adequate protection against moisture.

Moreover, the production of the fixed combination according to the invention comprising component A and B can also be carried out by the production of two melt extrudates which are independent of one another, which are combined in an additional process step and subsequently filled, for example, into sachets or capsules or formulated to give tablets. Each individual production step can be characterized by the addition of suitable excipients. After melt extrusion has taken place, comminution of the melt extrudates follows, preferably to a particle size of less than 0.315 mm. The granules thus obtained are mixed. The granule mixture can be directly packed, for example into sachets or capsules, or compressed to give tablets. Excipients such as already described can be employed. Independently of the final formulation, if necessary, attention is to be paid to appropriate protection against moisture.

Moreover, this invention also relates to the production of a fixed combination comprising component A and B by joint melt extrusion of both active substances in an active substance/excipient mixture. After melt extrusion has taken place, comminution of the melt extrudate follows, preferably to a particle size of less than 0.315 mm. The granules thus obtained can be packed directly, for example into sachets or capsules, or compressed to give tablets. Excipients such as already described can be employed. Independently of the final formulation, if necessary, appropriate protection against moisture is to be guaranteed.

The presentation forms produced comprising the fixed combination according to the invention can be provided with a coating in the last production step. Coatings within the meaning of this invention can be sugar coatings and in particular film coatings. The process follows generally standard processes and makes the addition of further excipients necessary. For the film coatings, film-forming agents are used such as, for example, modified celluloses, polymethacrylates, polyvinylpyrrolidone and polyvinyl acetate phthalate, plasticizers such as, for example, polyethylene glycols, triacetin, glycerol, dibutyl phthalate, colourants/pigments such as, for example, titanium dioxide, iron oxide and/or mould release agents such as, for example, talc, highly disperse silica, silica gel, magnesium stearate, glycerol monostearate. Coating is carried out by spraying on a suspension, preferably based on water. Replacement of the water by a suitable organic solvent is possible.

For construction as a capsule, both the customary hard gelatin capsules and hard capsules consisting of HPMC (methylhydroxypropylcellulose) are suitable. The hard HPMC capsules can be employed directly or after drying. Surprisingly, both the combination according to the invention and the individual components A and B are stable not only in the hard HPMC capsules but also in the hard gelatin capsules. This demonstrates again the stability of the combination according to the invention and medicament. Furthermore, hard capsules of other polymers (e.g. starch, cellulose, hydroxypropylcellulose, hydroxypropylcellulose lactate, hydroxypropylcellulose glycolide and hydroxyethylhydroxypropylcellulose) are suitable.

In particular, component B can be processed in crystalline or amorphous, ground or micronized form. In order to accelerate the absorption from the gastrointestinal tract, the preferred particle size of the component B employed within the context of this invention is an average particle size of X₅₀≦15 μm, preferably X₅₀≦8 μm (see FIG. 1: Example of a particle size distribution of micronized simvastatin).

For oral administration, known administration forms releasing the active substance complex rapidly and/or in modified form are suitable, such as, for example, tablets (uncoated and coated tablets, enteric coatings, ODT (oral dissolving tablets), effervescent tablets, chewable tablets), capsules, sugar-coated tablets, granules, pellets or powders.

The fixed combination according to the invention is preferably a formulation rapidly releasing the active substances (see FIG. 2: Example of release profiles of a tablet comprising the fixed combination of D,L-lysine o-acetylsalicylate and simvastatin). “Rapid-release formulation” is to be understood here as meaning a formulation of the type (e.g. tablet, capsule, sachet) which releases at least 80% of its contained active substance doses within 30 minutes (blade-stirrer apparatus according to USP; UV detection).

The fixed combination within the meaning of this invention can be administered up to three times daily; a combination which allows administration 1 to 2 times daily is preferred.

Surprisingly, the fixed combination according to the invention comprising component A and B is distinguished by a high stability. Significant decomposition of component A and/or B is not observed.

### Use:

The combination according to the invention can be employed for the prevention of cardiovascular diseases. Prevention is understood as meaning both primary and secondary prevention. Primary prevention is understood in this connection as meaning the protection of patients from a first cardiovascular disease which results in organ damage. Secondary prevention is understood in this connection as meaning the protection of patients who have already suffered organ damage as a result of a cardiovascular disease from a fresh cardiovascular disease.

Preferably, the combination according to the invention for the prevention of cardiovascular diseases is employed in patients who have an increased risk of developing a cardiovascular disease.

The patient group at increased risk mentioned here includes, for example, patients with micro- and macroangiopathy, patients with increased blood pressure (hypertension), patients with an increased risk of developing high blood pressure, patients with renal dysfunction such as, for example, mild renal heart failure (mild renal failure (MRF)), who have an increased creatinine level in the blood plasma, patients with diabetes, patients with disturbed glucose metabolism (prediabetic metabolic status), patients with an increased body mass index (BMI), patients with an increased homocysteine level, patients who have first-degree relatives who suffer or have suffered from a cardiovascular disease, and patients who have first-degree relatives who suffer from or have suffered from diabetes. An increased creatinine level is present, in particular, at a value of over 1.5 mg/dl in men and 1.4 mg/dl in women. An increased BMI is present, in particular, at a value of over 25 kg/m². In particular in patients with hypertension, an increased risk is present at a total cholesterol level of over 200 mg/dl and/or an LDL level (low density lipoprotein) of over 160 mg/dl. In particular in patients with diabetes, prediabetic metabolic status or renal dysfunction, an increased risk is present at a total cholesterol level of over 170 mg/dl and/or an LDL level (low density lipoprotein) of over 120 mg/dl. The patients with increased blood pressure include, in particular, patients with only slightly increased blood pressure (120/85 mmHg to 139/90 mmHg) and patients who, in the presence of increased blood pressure, have insufficiently reduced the increased blood pressure (>145/95 mmHg). An increased risk is present at an increased homocysteine level of 12 μmol/l in the blood.

Likewise, the patient group with increased risk mentioned here includes patients who have already suffered organ damage as a result of a cardiovascular disease. These patients already had, for example, a stroke, angioplasty, bypass operation, angina pectoris, diseases of the coronary vessels of the heart, myocardial infarct, thrombosis and/or pathological changes of the vascular wall.

Particularly preferably, the patients mentioned here with increased risk include patients with renal dysfunction such as, for example, mild renal heart failure (mild renal failure (MRF)), who have an increased creatinine level in the blood plasma, patients with only slightly increased blood pressure (120/85 mmHg to 139/90 mmHg) and patients who, in the presence of increased blood pressure, have insufficiently reduced the increased blood pressure (>145/95 mmHg).

The combination according to the invention shows, in patients who have an increased risk of developing a cardiovascular disease, an unexpectedly broad and versatile spectrum of action. Cardiovascular diseases are understood as meaning diseases such as micro- and macroangiopathy, arteriosclerosis, stroke, angina pectoris, diseases of the coronary vessels of the heart, in particular the arterial coronary vessels, heart failure, myocardial infarct, pathological changes of the vascular wall, circulatory disturbances, disturbances of the microcirculation, lipid metabolism disorders such as hyperlipidaemia, dyslipidaemia, increased concentration of lipoproteins in the serum and possibly a shift in the lipoprotein components, hyperlipoproteinaemia, increase both in the serum cholesterol and in the serum triglycerides combined with increased VLDL (very low density lipoprotein) and increase in the chylomicrons in the plasma, disturbances of the homocysteine level, non-insulin-dependent diabetes mellitus (=Type 2 diabetes), diabetes, hypoglycaemia, metabolic disturbances such as disturbance of the lipid metabolism, deficiency of acidic lipase, storage diseases, in particular lipid storage diseases, phytosterolaemia, high blood pressure, obesity, thromboses, pancreatitis, constipation (obstipation), functional disorders of the brain, cerebrovascular insufficiency, cerebral circulatory disorders, apoplexy, transitory ischaemic attacks (TIA), dementia and fainting.

Of particular interest is the use of the combination according to the invention in “cardiac risk management”, i.e. in the prophylaxis of cardiovascular diseases which are influenced or caused by more than one risk factor such as, for example, arteriosclerosis, diseases of the coronary vessels of the heart, in particular the arterial coronary vessels, raised serum lipids, hypercholesterolaemia, hypertriglyceridaemia, increase both in the serum cholesterol and the serum triglycerides combined with increased VLDL (very low density lipoprotein) and increase in the chylomicrons in the plasma and syndrome X. Typical risk factors are increased cholesterol levels, reduced HDL levels, smoking, glucose intolerance and cardiomegaly. The risk factors can vary depending on the age and sex of the patient.

“Hyperlipidaemia” is to be understood as meaning an increased plasma level of one or more serum lipids. In this respect, the LDL level is particularly of importance. Increased levels are regarded in patients of over 45 years as values over 130 mg/dl and in patients of under 45 years as values over 160 mg/dl.

“Dyslipidaemia” is to be understood here as meaning hypertriglyceridaemia or hypercholesterolaemia, but particularly a mixed hyperlipidaemia, i.e. a disease state with an increased cholesterol level (LDL and total cholesterol) and increased triglyceride level. This can be associated with a decrease in the HDL (high density lipoprotein) cholesterol in the plasma or a disturbed HDL-C/LDL-C ratio.

The combination according to the invention proves to be surprisingly advantageous in the prevention of micro- and macroangiopathy, coronary heart diseases, cardiac insufficiency, disturbance of the brain capacity, apoplexy, circulatory disorders, disturbances of the lipid metabolism, high blood pressure or of diabetes mellitus.

The effect is particularly severe in patients with micro- or macroangiopathy, in particular as a result of high blood pressure, patients with high blood pressure, patients with renal dysfunction such as, for example, mild renal heart failure (mild renal failure (MRF)), patients who have an increased creatinine level in the blood plasma, patients with diabetes and patients with a disturbed glucose metabolism (prediabetic metabolic status).

In particular, it has been found that the combination according to the invention leads to surprising effects in patients who have an increased risk of developing a cardiovascular disease, but have a normal cholesterol level, who have an only slightly increased blood pressure (120/85 mmHg to 139/90 mmHg) or who, in the presence of an increased blood pressure, have reduced the increased blood pressure insufficiently (RR>145/95 mmHg).

When using the combination according to the invention, a synergistic effect which is not to be expected is observed in the action. Thus, the amounts of the active substances employed in the combination can be reduced in comparison to monotherapy.

The synergistic effect of the combination according to the invention is preferably observed if the combination according to the invention contains 0.1 to 20 mg/kg, in particular 0.5 to 5 mg/kg, of active substance of component A (calculated as o-acetylsalicylic acid), 0.001 to 10 mg/kg, in particular 0.005 to 5 mg/kg, of active substance of component B, in each case relative to kg of body weight of the patient on oral administration.

In addition, the synergistic effect of the combination according to the invention is preferably observed if the combination according to the invention contains as component A the salt of o-acetylsalicylic acid with lysine in a dose of 25 to 500 mg, preferably in a dose of 50 to 350 mg, particularly preferably in a dose of 75 to 200 mg (calculated as o-acetylsalicylic acid) and as component B atorvastatin in a dose of 1 to 200 mg, preferably in a dose of 5 to 80 mg, particularly preferably in a dose of 20 to 40 mg, simvastatin in a dose of 2.5 to 160 mg, preferably in a dose of 5 to 80 mg, particularly preferably in a dose of 10 to 40 mg, pravastatin in a dose of 2 to 100 mg, preferably in a dose of 5 to 80 mg, particularly preferably in a dose of 20 to 40 mg, or lovastatin in a dose of 5 to 200 mg, preferably in a dose of 5 to 80 mg, particularly preferably in a dose of 10 to 40 mg.

The synergistic effect of the combinations according to the invention is preferably observed if the components A and B of the combinations according to the invention are present in a ratio of 2:1 to 100:1, preferably 2:1 to 10:1, particularly preferably 2.5:1 to 5:1 with respect to A (calculated as o-acetylsalicylic acid) and B.

“Ratio” within the meaning of the invention is understood as meaning the weight ratio of the individual components.

Optionally, it may be necessary to depart from the amounts mentioned, namely depending on the body weight or on the type of administration route, on the individual behaviour towards the medicaments, the manner of their formulation and the time or interval at which administration takes place. Thus in some cases it may be adequate to manage with less than the abovementioned minimum amount, while in other cases the upper limit mentioned has to be exceeded. In the case of the administration of relatively large amounts, it may be advisable to divide these into a number of individual doses over the course of the day.

The combination according to the invention is furthermore distinguished by surprisingly good tolerability.

As component A, additionally the salt of o-acetylsalicylic acid with a basic amino acid, preferably lysine, can also be employed which, in a particle size distribution measured using a Malvern 2600D apparatus under standard conditions, has an average particle size above a particle size of 160 μm and a proportion of more than 60% of the particles with a particle size in a range from 100 to 200 μm, preferably an average particle size above a particle size of 170 μm and a proportion of more than 70% of the particles with a particle size in a range from 100 to 200 μm. Component A customarily has a residual moisture content of less than 0.4%, preferably of less than 0.3% and in particular of less than 0.15% of water. The low residual moisture content leads to an improved stability of component A and consequently also of the combination according to the invention and of the medicament comprising this combination. The preparation process is described in WO 02/005782.

A further embodiment of component A is an active substance complex consisting of a salt of o-acetylsalicylic acid with a basic amino acid, preferably lysine, and glycine. The active substance complex and its production is described in the German patent application DE 102005025283. The content of glycine in the active substance complex is 8 to 12, preferably 9 to 11, particularly preferably 10 percent by weight based on the active substance complex. The advantageous properties of the present invention are observed independently of the particle size of the active substance complex. The particle size distribution of the active substance complex has, for example, an average particle size of preferably below 100 μm, particularly preferably of below 70 μm.

The active substance complex consisting of a salt of o-acetylsalicylic acid with a basic amino acid and glycine is described in German application DE 102005025283 and can be prepared as follows:

o-Acetylsalicylic acid and the appropriate amino acid are mixed together as rapidly as possible, preferably in less than 20 minutes, at a temperature of less than or equal to 40° C., preferably from 20 to 35° C., under normal pressure and mixed to give a homogeneous phase, such that the temperature does not exceed 40° C. Seed crystals are optionally added to the homogeneous mixture prepared in this way, it is cooled to −5 to 10° C., preferably to 0 to 5° C. and stirred at this temperature for 2 to 8 hours, preferably 3 to 5 hours. It is treated with cooled acetone and with the amount of optionally cooled glycine needed. The suspension should be kept under the conditions indicated above for at least one hour for completion of the crystallization. According to the invention, a crystallization period of 1 to 10 hours under the conditions indicated above is preferred, a period of 1 to 8 hours being particularly preferred. It is very important according to the present invention that the temperature during the crystallization process is kept within limits which are as narrow as possible. The temperature must not exceed 5° C. and should preferably be kept below 3° C., particularly preferably between 0 and 2° C. Crystals of the desired product can be used as seed crystals. The crystallization is preferably carried out under normal pressure.

The crystallizate is subsequently isolated in the conventional manner, for example by filtering or centrifuging. The solid is washed a number of times with organic solvents, according to the invention alcohols such as, for example, ethanol and/or ketones such as acetone or mixtures of alcohols and/or ketones, for example mixtures of ethanol and acetone, or the use of various solvents of this type being preferred.

The solid is subsequently dried under reduced pressure. Here, the temperature should be kept below 50° C., preferably below 40° C. and particularly preferably below 35° C. A pressure of less than 100 mbar, preferably of less than 50 mbar should be applied to the solid. Drying can be carried out under conventional conditions, for example in a drying apparatus.

Suitable solvents for the reaction partner are water or water-miscible organic solvents such as, for example, alcohols such as methanol, ethanol or isopropanol, in particular ethanol, ethers such as tetrahydrofuran (THF) or ketones such as acetone, or mixtures of the solvents mentioned. Water, ethanol or a mixture of the two is preferred.

Preferably, the o-acetylsalicylic acid is dissolved in ethanol and the amino acid, preferably lysine, particularly preferably D,L-lysine monohydrate dissolved in water, is mixed together.

The reaction partners are employed in amounts of the type such that the basic amino acid is present in a slight excess based on the moles of o-acetylsalicylic acid. According to the invention, a molar ratio of o-acetylsalicylic acid to amino acid of 1:1.05 to 1:1.5 is preferred, a ratio of o-acetylsalicylic acid to amino acid of 1:1.05 to 1:1.2 being particularly preferred.

According to the present invention, the o-acetylsalicylic acid should have a content of 1 to 10% by weight, preferably 5 to 10% by weight and particularly preferably of 6 to 8%, by weight of o-acetylsalicylic acid. The solution of the basic amino acid should have a content of 10 to 40% by weight, preferably 15 to 35% by weight and particularly preferably of 20 to 30% by weight, of amino acid.

The glycine can be added according to the present invention as a solution in water or a water-miscible organic solvent, where the solvents described above can be employed as an organic solvent, to the reaction mixture of the reaction partners.

The glycine can be added according to the present invention, but also in the form of a suspension. The glycine suspension can be prepared in a conventional manner. According to the invention the preparation of a glycine suspension from a solvent mixture of water and an alcohol such as, for example, ethanol is preferred.

Likewise important in the process according to the invention is the adherence to a certain stirring energy during the crystallization. The homogeneous mixture of the starting materials must only be stirred gently. The stirring energy to be applied should not be greater than 0.1 W per litre of reaction medium. According to the invention, an applied stirring energy of 0.04 to 0.06 W per litre of reaction medium is preferred. Possible stirrers are all conventional appropriately regulatable stirring apparatuses such as, for example, a stirrer container with a flow disruptor.

The combination according to the invention is preferably employed in human medicine, but is also suitable for veterinary medicine, in particular for the treatment of mammals.

### WORKING EXAMPLES

The present invention is shown in more detail below by means of non-restrictive preferred examples (1 kg scale; dose unit 450 mg). If not indicated otherwise, all quantitative data relate to percentages by weight and ratios to ratios by weight.

### Example 1

402.9 g of D,L-lysine o-acetylsalicylate are mixed with 68.4 g of mannitol, 141.1 g of microcrystalline cellulose, 112.9 g of sodium carboxymethylcellulose, 12.0 g of highly disperse silica and 24.2 g of magnesium stearate and compacted by means of dry granulation. The resulting briquettes are screened in 2 stages.

106.7 g of microcrystalline cellulose, 48.9 g of mannitol, 7.8 g of sodium carboxymethylcellulose, 44.4 g of micronized simvastatin, 6.7 g of hydroxypropylmethylcellulose, 1.1 g of sodium laurylsulphate and 3.3 g of ascorbic acid are wet-granulated and the resulting granules are screened once.

The two granulates prepared are combined, homogenized and subsequently mixed with 19.6 g of magnesium stearate. Direct filling of the granules or compression to give tablets takes place. Coating of the pharmaceutical form can optionally be carried out.

### Example 2

444.4 g of active substance complex D,L-lysine o-acetylsalicylate with glycine from Example 5 and 44.4 g of micronized simvastatin are mixed with 90.0 g of mannitol, 244.4 g of microcrystalline cellulose, 120.7 g of sodium carboxymethylcellulose, 12.2 g of highly disperse silica and 24.2 g of magnesium stearate and compacted by means of dry granulation. The resulting briquettes are screened in 2 stages. After subsequent mixing of the granules with 19.6 g of magnesium stearate, direct filling of the granules or compression to give tablets takes place. Coating of the pharmaceutical form can optionally be carried out.

### Example 3

402.9 g of D,L-lysine o-acetylsalicylate are mixed with 149.9 g of microcrystalline cellulose, 68.4 g of mannitol and 112.9 g of sodium carboxymethylcellulose.

44.4 g of micronized simvastatin, 44.4 g of mannitol and 133.2 g of hydroxypropylcellulose are mixed, extruded and the extrudate obtained is comminuted (particle size<0.315 [mm]).

The powder mixture with D,L-lysine o-acetylsalicylate is homogenized with the simvastatin extrudate granules, subsequently mixed with 43.8 g of magnesium stearate and directly filled or compressed to give tablets. Coating of the pharmaceutical form can optionally be carried out.

### Example 4

As Example 1, but after compression of the granules to give tablets, lacquering of the tablets with an aqueous Opadry II suspension (number 85G), a moisture-protective lacquer based on PVA, takes place. For lacquering, 30.0 g of Opadry II (number 85G) are needed.

### Example 5

Active Substance Complex of D,L-lysine o-acetylsalicylate with 10% of Glycine

In a sterile and pyrogen-free stirrer container with a flow disruptor, a pyrogen-free solution of 40.0 kg of o-acetylsalicylic acid in 500 kg of ethanol is added through a sterile filter. At 20 to 30° C., a sterile-filtered and pyrogen-free solution of 36.4 kg of D,L-lysine monohydrate in 110 kg of pyrogen-free water is added with stirring and cooling in the course of a short time (under 15 minutes), such that a temperature of 35° C. is not exceeded. At least 20 g of sterile seed crystals are added and the already crystallizing mixture is cooled to 2° C. with reduced speed of rotation of the stirrer. 490 kg of pyrogen-free and temperature-controlled acetone and a suspension of 8.0 kg of glycine prepared aseptically and under temperature-controlled conditions in 25.0 kg of pyrogen-free water and 90 kg of ethanol are subsequently added. With further cooling at 2° C., the suspension is stirred further for 1 to 8 hours. Only then is the crystal mixture isolated under aseptic conditions in a filter or a centrifuge. The moist product is washed with pyrogen-free ethanol and acetone in the separation apparatus and dried under aseptic conditions at a pressure of ≦50 mbar and a temperature of not more than 40° C. Subsequently, the finished product is filled into containers with PE inliners and sealed. 60 to 70 kg (75 to 87% of theory) of the title product with a residual moistness of <0.3% are obtained with an average particle size of 41 μm.

### Melting Point Determination of Example 5 by DSC (Differential Scanning Calorimetry):

The melting point determinations by DSC are carried out with the Pyris-1 apparatus of Perkin Elmer at a heating rate of 20 K/min. Dry nitrogen is used as a protective gas. The characteristic DSC curves of product according to Example 5 show two peaks, an endothermic peak at 147.9±1.44° C. followed by an exothermic peak at 153.0±1.0° C. The endothermic peak results from the melting process, whereas the endothermic peak results from an overlapping of decomposition and partial crystallization of a decomposition product (e.g. of o-acetylsalicylic acid) in the molten phase.

### Stability Data

The tablets are packed in brown glass bottles with drying agent in the cover and stored in climate chambers at 25° C./60% relative humidity and 40° C./75% relative humidity.

The tablets are regularly removed from storage and analysed for their content of acetylsalicylic acid, salicylic acid as a degradation product of acetylsalicylic acid, simvastatin and hydroxy acid as a degradation product of simvastatin. The analysis method employed is a selective HPLC method.

The tablets investigated are prepared as described above and are defined in the following manner:

ASA-SIMVA TABL 100+20 MG contains 100 mg of o-acetylsalicylic acid and 20 mg of simvastatin,

ASA LYS-SIMVA TABL 100+20 MG contains 181 mg of D,L-lysine o-acetylsalicylate (correspond to 100 mg of o-acetylsalicylic acid) and 20 mg of simvastatin,

ASA LYS GLY-SIMVA TABL 100+20 MG contains 200 mg of active substance complex D,L-lysine o-acetylsalicylate with glycine (Example 5) (correspond to 100 mg of o-acetylsalicylic acid) and 20 mg of simvastatin.

TABLE 1
Content of o-acetylsalicylic acid (mg/tablet)

| | | 3 months, 25° C./ | 3 months, 40° C./ |
|---|---|---|---|
| | Start | 60% rel. humidity | 75% rel. humidity |
| ASA - SIMVA TABL | 95 | 93 | 28 |
| 100 + 20 MG | | | |
| ASA LYS - SIMVA | 95 | 94 | 90 |
| TABL 100 + 20 MG | | | |
| ASA LYS GLY - SIMVA | 96 | 95 | 93 |
| TABL 100 + 20 MG | | | |

TABLE 2
Salicylic acid (% based on declared content of acetylsalicylic
acid) as a degradation product of acetylsalicylic acid

| | | 3 months, 25° C./ | 3 months, 40° C./ |
|---|---|---|---|
| | Start | 60% rel. humidity | 75% rel. humidity |
| ASA - SIMVA TABL | 0.9 | 3.0 (Δ 2.1%) | 18.8 (Δ 17.9%) |
| 100 + 20 MG | | | |
| ASA LYS - SIMVA | 3.3 | 4.6 (Δ 1.3%) | 8.8 (Δ 5.5%) |
| TABL 100 + 20 MG | | | |
| ASA LYS GLY - SIMVA | 2 | 2.8 (Δ 0.8%) | 5.0 (Δ 3.0%) |
| TABL 100 + 20 MG | | | |

Δ: difference to the starting value

TABLE 3
Results: Content of simvastatin (mg/tablet)

| | | 3 months, 25° C./ | 3 months, 40° C./ |
|---|---|---|---|
| | Start | 60% rel. humidity | 75% rel. humidity |
| ASA - SIMVA TABL | 23 | 20 | 13 |
| 100 + 20 MG | | | |
| ASA LYS - SIMVA | 21 | 21 | 21 |
| TABL 100 + 20 MG | | | |
| ASA LYS GLY - SIMVA | 22 | 21 | 22 |
| TABL 100 + 20 MG | | | |

TABLE 4
Hydroxy acid (% based on declared content of simvastatin)
as a degradation product of simvastatin

| | | 3 months, 25° C./ | 3 months, 40° C./ |
|---|---|---|---|
| | Start | 60% rel. humidity | 75% rel. humidity |
| ASA - SIMVA TABL | 0.6 | 0.8 (Δ 0.2%) | 4.4 (Δ 3.8%) |
| 100 + 20 MG | | | |
| ASA LYS - SMVA | 0.3 | 0.2 (Δ - 0.1%) | 0.5 (Δ 0.3%) |
| TABL 100 + 20 MG | | | |
| ASA LYS GLY - SIMVA | 0.3 | 0.1 (Δ - 0.2%) | 0.4 (Δ 0.1%) |
| TABL 100 + 20 MG | | | |

Δ: difference to the starting value

### RESULT

The tablets according to the invention (ASA LYS-SIMVA TABL 100+20 MG and ASA LYS GLY-SIMVA TABL 100+20 MG) comprising a fixed combination of D,L-lysine o-acetylsalicylate or its active substance complex with glycine, and simvastatin, which are present together in the same layer in a common matrix, show a higher stability than the comparison example comprising o-acetylsalicylic acid and simvastatin (ASA-SIMVA TABL 100+20 MG).

1. A composition comprising a salt of o-acetylsalicylic acid with a basic amino acid as component A and an HMG-CoA reductase inhibitor as component B.

2. The composition according to claim 1 wherein the HMG-CoA reductase inhibitor is simvastatin.

3. The composition according to claim 1, wherein the basic amino acid is lysine.

4. The composition according to claims 1, wherein component A further comprises glycine.

5. The composition according to claim 4, wherein component A comprises 8 to 12 percent by weight of glycine.

6. The composition according to claim 4, wherein component A comprises 10 percent by weight of glycine and has an endothermic peak temperature of 148±2° C. and an exothermic peak temperature of 153±2° C.

7. A pharmaceutical composition comprising the composition according to claim 1 and one or more further suitable excipients.

8. The pharmaceutical composition according to claim 7, wherein component A and B of the composition are stably present together in a joint matrix in the same layer.

9. A method of making the pharmaceutical composition according to claim 7, wherein components A and B are mixed and directly filled or tabletted.

10. The method according to claim 9, wherein components A and B are dry-granulated together and subsequently directly filled or tabletted.

11. The method according to claim 9, wherein component A is dry-granulated, component B is wet-granulated, components A and B are mixed to form granules, which are then directly filled or tabletted.

12. The method according to claim 9, wherein component A is used in the form of a powder mixture and component B is used in the form of extrusion granules.

13. The method according to claim 9, wherein component A and component B are used as two extrusion granulates independent of one another.

14. The method according to claim 9, wherein components A and B are used as a common extrusion granulate.

15 A method of treating a cardiovascular disease in a subject by administering a therapeutically effective amount of the composition of claim 1.

16. The method according to claim 15, wherein the subject has an increased risk of developing a cardiovascular disease.

17. A method of treating a cardiovascular disease in a subject by administering a therapeutically effective amount of the pharmaceutical composition of claim 7.
